(19) United States
(12) Patent Application Publication
(10) Pub. No.: US 2024/0050090 A1
(43) Pub. Date: Feb. 15, 2024

(54) SINGLE-IMPULSE, SEMI-MOBILE AND STOPPERED SUTURING DEVICE USED IN NARROW AND DEEP TISSUE PLANES SURGERY

(71) Applicant: SORANUS ARGE VE DANISMANLIK HIZMETLERI SANAYI TICARET A.S., Izmir (TR)

(72) Inventor: Ahmet Ozgur Yeniel, Izmir (TR)

(73) Assignee: SORANUS ARGE VE DANISMANLIK HIZMETLERI SANAYI TICARET A.S., Izmir (TR)

(21) Appl. No.: 18/383,394

(22) Filed: Oct. 24, 2023

Related U.S. Application Data
(63) Continuation of application No. 16/979,909, filed on Sep. 11, 2020, now abandoned.

(30) Foreign Application Priority Data
Mar. 23, 2018 (TR) 2018/04159

(51) Int. Cl. A61B 17/062; A61B 17/28; A61B 17/29

(52) U.S. Cl. CPC A61B 17/0625; A61B 17/282; A61B 17/29; A61B 2017/00473

(57) ABSTRACT

The present invention relates to a surgical device that is defined as a single-impulse suturing device, which allows both suturing and grasping a needle at the opposite end for one time without any need for another tool to retrieve the needle after being passed through a tissue during the suturing process, and it comprises the following characteristics of; allowing a safe and minimal invasive access to narrow and deep surfaces, performing a well-directed suturing from a determined point due to its semi-mobile jaw structure, having a stopper that enables suturing by staying at a distance from neurovascular structures in an invisible area, and allowing a multiple suturing with the same needle. The invention keeps suturing function of the single-impulse devices reserved and develops the same in addition to combining this function with ergonomic size characteristics and practical handle advantages of double-impulse devices.

### TECHNICAL FIELD OF THE INVENTION

The present invention relates to a surgical device that has been developed to be used in repair of the tissue structures with access difficulties due to their narrow and deep localizations, which lack anatomical integrity due to ruptures, breakages or other various reasons.

### PRIOR ART

Numerous prior art surgical devices used in surgical repair of the tissue structures lack anatomical integrity due to ruptures, breakages or other various reasons have been provided.

In the prior art, several devices in various sizes and types regarding the single-impulse suturing in surgical repair have been provided. Suturing is a process of attaching a thread to the tissues intended to be faced together by means of a needle and joining them together by sewing. If the needle needs to be received from the opposite side with a second stage in case of sewing, it is defined as double-impulse suturing. Single-impulse suturing is, on the other hand, defined as a process of both stitching in one time and grasping the needle at the opposite end without any need for any other second stage in order to retrieve the needle after it is being passed through the tissue. Suturing on the surfaces with narrow and deep anatomical locations contains a risk due to their vicinity to the close vessels, nerves or visceral organs. Anatomical information of these structures is a must for safe surgery. The distances in suturing on narrow and deep surfaces in the prior art are determined by finger measurement, based on their anatomical data. Yet, there has not been provided a mechanism that enables objective measurement by using determined anatomical triangulation points as a guide.

### SUMMARY AND AIMS OF THE INVENTION

The invention is capable of providing the advantages that the prior art suturing devices do not have when intervention is challenging or use of invasive methods is a must if there is a surgical repair indication, in particular due to narrow and deep anatomical localizations.

The shorter the excluding process of the tissues is, the lesser the surgical intervention is invasive or minimally invasive.

The suturing device according to the present invention has a structure so as to be easily brought to the target between two fingers, in order to allow processing with minimal invasion during access to the surfaces located in narrow and deep localizations, wherein it is needed to pass through an inclined path and in their vicinity, neurovascular structures are present.

The most prominent aim of the invention is to provide a suturing device that has a suitable size and inclination for minimal invasive use depending on the anatomical structure in narrow and deep localizations, and is also capable of single-impulse suturing and sewing through a safe surface with a device having a stopper mechanism in addition to suturing so as to enable a second suturing in a multiple manner, in other words, with the same needle.

By means of the invention, after the single-impulse suturing needle is passed through the tissue, both suturing for one time and grasping the needle at the opposite end is enabled without any need for another device to retrieve the needle.

An aspect of the invention for suturing on a safe surface includes providing a safe range that allows protection against anatomically well-defined structures such as vessels and nerves with the help of a stopper mountable relative to the location of the target tissue to the stationary one of the arms at the jaw part to which the suture is added. Thus, the fixed jaw stopper allows the point for suturing to be safe regarding the neurovascular vicinity based on the anatomical triangulation point.

A second advantage of the suturing device according to the invention is that an end portion of the device is adaptable depending on thickness of the tissue to be sutured when the target is achieved.

Another advantage of the invention is that it has a structure compatible with the need of the device for multiple suturing through the same surface. Multiple suturing may be necessary for stronger gripping in addition to some operational techniques.

A further important advantage of the suturing device according to the invention is that a mechanism that is located at a handle portion and allows suturing can be easily controllable by the other hand not holding a clipper.

As both hands are located at an ideal distance when using the, this makes it easier to perform suturing with the device and it also contributes to the minimal invasive approach of the device. In order to be able to adjust the ideal distance, double impulse suturing devices can be considered as well. In such a case, the similarity between the size of the ideal device with the formation of the handle portion and the devices used in conventional surgery or double impulse suturing techniques would be important. Double impulse suturing devices have a nature convenient for end controlling from the handle. Single impulse devices have emerged as a result of some handicaps such as showing the target tissue that is a must in use of these devices and use of retractors containing comprehensive dissections and traumatic processes.

The suturing device according to the invention brings together the aspect of corresponding size of double impulse devices and their advantages of having a practical handle with the suturing function of single impulse devices.

In the prior art, there has not been provided a single impulse suturing device;
located in a narrow and deep localization, having sizes and an inclination suitable to the surfaces that can be reached after passing through an inclined path,
in a nature adjustable relative to thickness of an end portion of a tissue
facilitating more than one suturing, and
which is capable of performing the processes together including easy controlling of a handle mechanism with the other hand.

### BRIEF DESCRIPTION OF THE DRAWINGS

The drawings used to depict the suturing device developed with the present invention more explicitly, and the corresponding definitions of these drawings are as follows.

FIG. 1: a perspective view of a suturing device

FIG. 2: a perspective view of a jaw portion of the suturing device

FIG. 3-7: detailed views from different perspectives of the jaw portion of the suturing device

FIG. 8-11: views in case that single impulse suturing is performed through a tissue with the suturing device

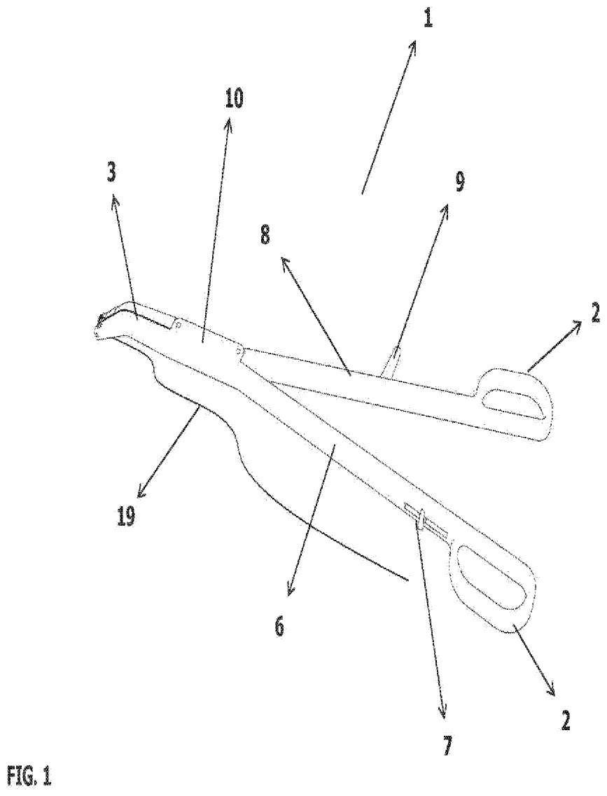

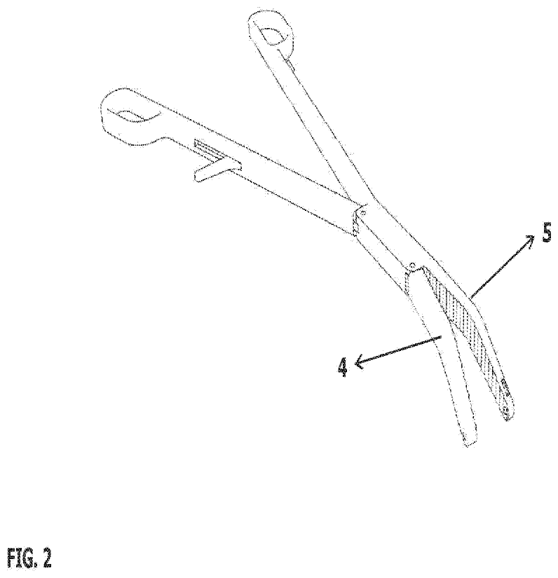

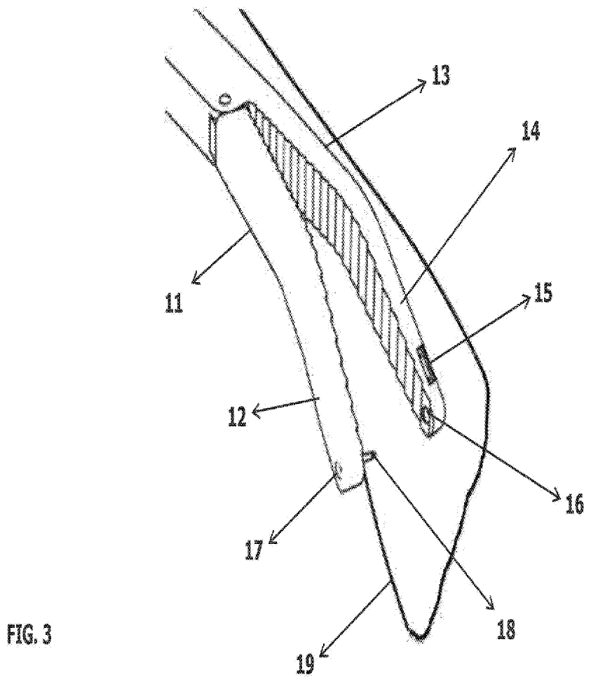

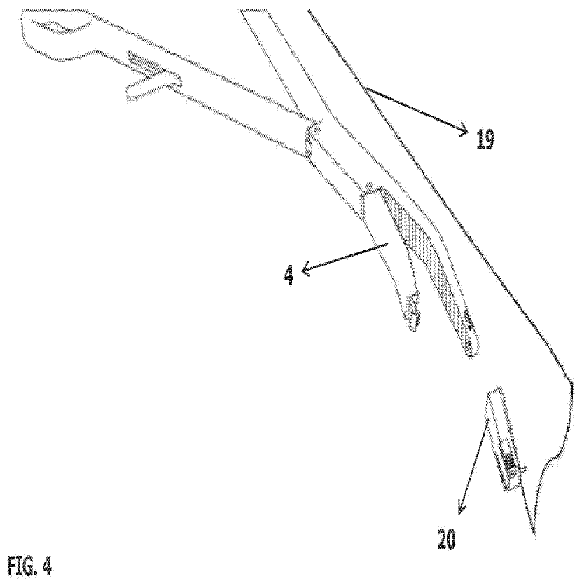

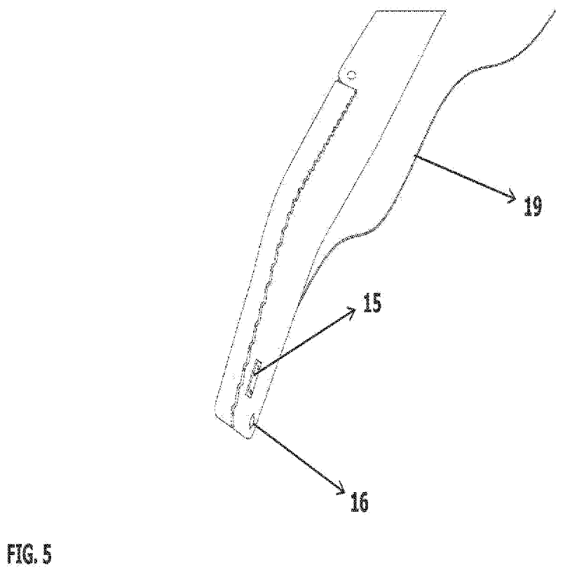

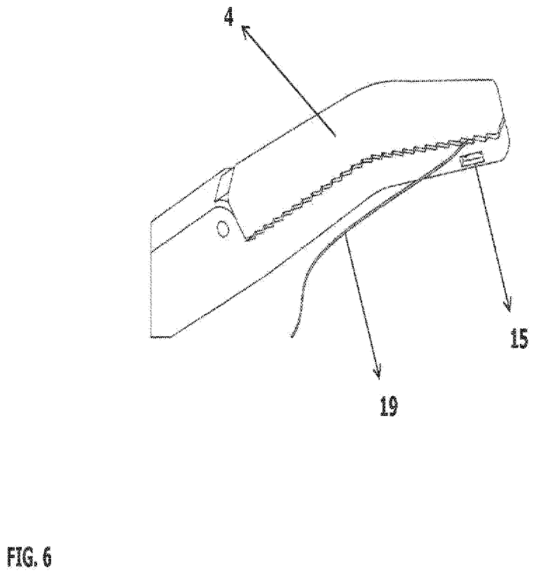

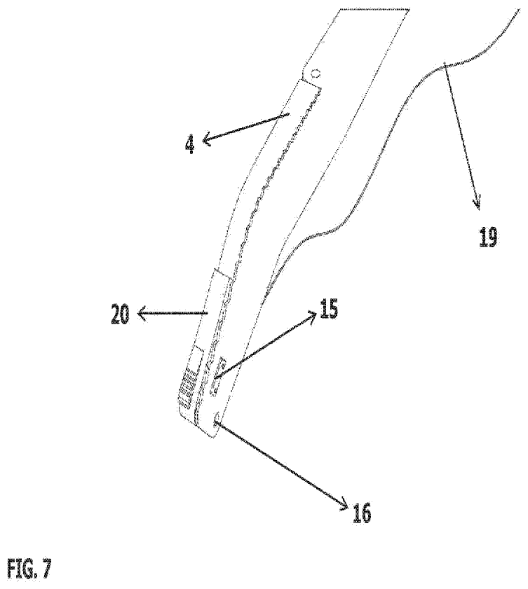

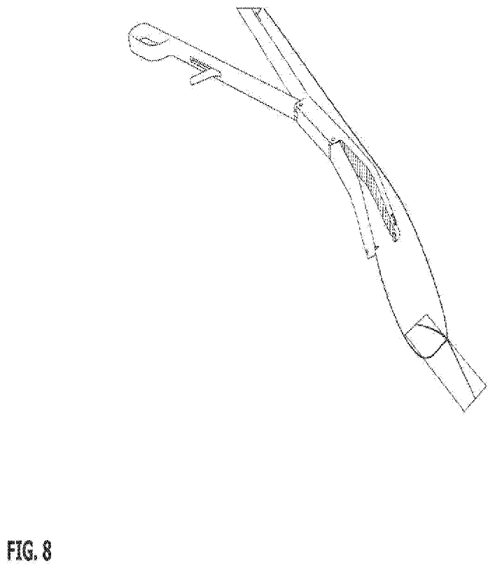

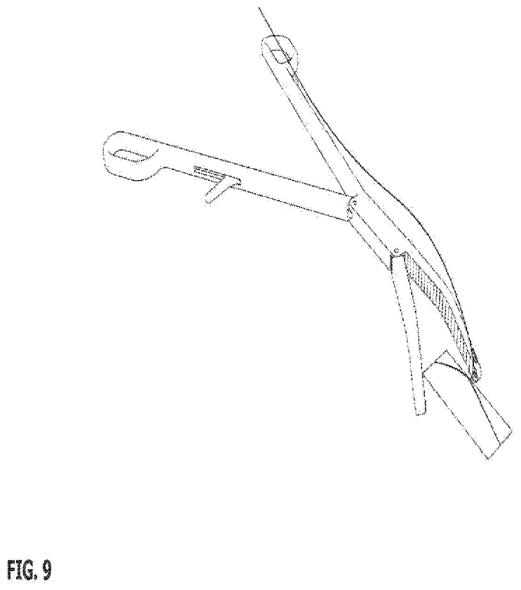

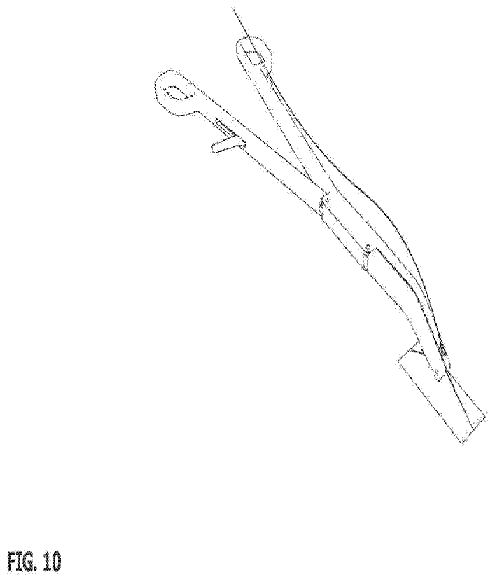

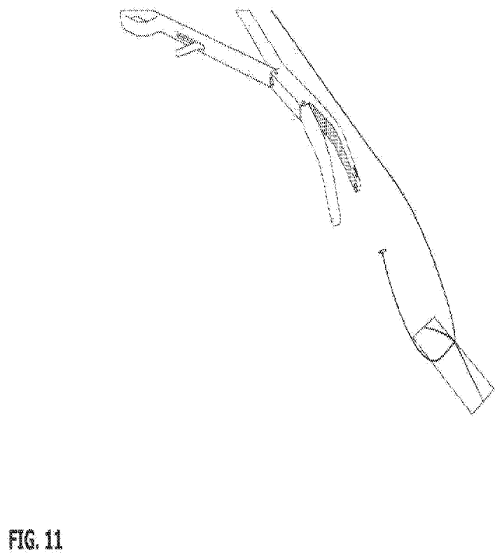

### DEFINITIONS OF THE COMPONENTS/PORTIONS/PARTS FORMING THE INVENTION

The parts and components presented in the drawings are separately numbered in order to depict the suturing device developed according to the invention more evidently and the corresponding definition of every number is as follows.
1. Suturing device
2. Holding tab
3. Jaw assembly
4. Movable jaw
5. Fixed jaw
6. Fixed jaw conveying arm
7. Fixed jaw mechanism control pin
8. Movable jaw conveying arm
9. Fixed jaw mechanism control pin
10. Conveying arm body
11. Movable jaw base
12. Movable jaw end
13. Fixed jaw base
14. Fixed jaw end
15. Stopper partition
16. Fixed jaw needle grasping slot
17. Movable jaw needle grasping slot
18. Needle
19. Thread
20. Movable jaw end attachment

### DETAILED DESCRIPTION OF THE INVENTION

The suturing device (1) according to the invention is developed to be used in repair of the tissue structures with access difficulties due to their narrow and deep localizations, which lack anatomical integrity due to ruptures, breakages or other various reasons; comprising
at least two holding tabs (2) that allow the operator to grasp the suturing device (1) and a jaw assembly (3) to be opened and closed,
a jaw assembly (3) formed of a fixed jaw (5) containing a movable jaw (4) having one needle grasping slot (17) and one needle (18) on a jaw end (12), and a fixed jaw (5) having one stopper partition (15) and one needle grasping slot (16) on a jaw end (14),
a fixed jaw arm (6) that enables the fixed jaw (5) to be controlled via the holding tab (2) and is coupled to the fixed jaw (5) through a carrying arm body (10),
a fixed jaw mechanism control pin (7) that allows the mechanism at the fixed jaw end (14) to be controlled by passing through the fixed jaw carrying arm (6), and also allows a needle (18) to which a thread (19) is attached to be transferred between the movable and fixed jaw needle grasping slots (17 and 16),
a fixed jaw carrying arm (8) that allows the movable jaw (4) to be controlled via the holding tab (2) and is coupled to the movable jaw (4) through the carrying arm body (10),
a movable jaw mechanism control pin (9) that allows the mechanism at the movable jaw end (12) to be controlled by passing through the movable jaw carrying arm (8), and also allows a needle (18) to which a thread (19) is attached to be transferred between the movable and fixed jaw needle grasping slots (17 and 16),
a carrying arm body (10) in which the fixed jaw carrying arm (6) and the movable jaw carrying arm (8) are joined,
a movable jaw base (11) and a fixed jaw base (13) that have a flat geometry so as to be closed on a horizontal plane when they correspond to each other,
a movable jaw end (12) and a fixed jaw end (14) that have a geometry so as to be closed by making an angle in the direction of the movable jaw base (11), relative to the movable jaw base (11) and the fixed jaw base (13) when they correspond to each other,
a stopper partition (15) located on the fixed jaw end (14) and to which a stopper can be attached,
a stopper that can be attached to the stopper partition (15) located on the fixed jaw end (14) and is used to allow the point to be sutured to be safe regarding the neurovascular vicinity based on the anatomical triangulation point,
a fixed jaw needle grasping slot (16) and a movable jaw needle grasping slot (17) that are located at the end portions of the movable and fixed jaws (4 and 5) and allow the needle (18) to which the thread (19) is attached to be secured in order to make the same ready for going in to the tissue, and
a needle (18) to which a thread is attached via the fixed jaw mechanism control pin (7) and the fixed jaw mechanism control pin.

Furthermore, in another embodiment of the invention, the movable jaw end can have a structure as a movable jaw end attachment (20) that can be kept by removing from the movable jaw (4) together with the needle (18), and when needed, can be used by being attached to the movable jaw (4) together with the needle (18) to which the thread (19) is attached.

Operational principle of the invention can be described as follows.

The jaw assembly (3) is opened by holding the holding tabs (2) of the device (1) in order to perform suturing. The needle (18) to which the thread (19) is attached is mounted on the movable jaw needle grasping slot (17) in which the needle (18) attached with the thread (19), which is located at the movable jaw end (12), will be placed by means of the movable jaw mechanism control pin (9) on the movable jaw carrying arm (8).

Then, the jaw assembly (3) is closed and thus the needle (18) attached with the thread (19) can be made ready for going in to the tissue in a secured manner by the movable jaw needle grasping slot (17) and the fixed jaw needle grasping slot (16).

The stopper having a determined length for the former place right before it goes to the target tissue is attached to the stopper partition (15). The stopper would have a structure that is sharp, do not contain any cutting element and has a flexible elasticity. The jaw assembly (3) and the stopper are placed in a closed state between two fingers, and thus it would be possible to reach to the target tissue by using inclination of the device (1).

When an edge of the stopper at a determined length is placed at the anatomical triangulation point by use of fingers, the point through which suturing is done would be determined. In order to suture, the jaw assembly (3) is opened in accordance with the thickness of the target tissue and the fixed jaw (5) is fixed at the stopper distance such that the target tissue is sandwiched between the jaws (4 and 5).

Then, the movable jaw (4) is closed, the needle (18) attached with the thread (19) passes through the tissue and the fixed jaw is placed in the needle grasping slot (16). Meanwhile, the needle (18) attached with the thread (19) would be transferred to the fixed jaw needle grasping slot (16) with the help of the control pins (7 and 9) placed on the carrying arms (6 and 8).

At this stage, when the jaw assembly (3) is opened and retracted a bit, the tissue is sutured and the jaw assembly (3) is closed in the gap and then the needle (18) is transferred to the movable jaw (4) via the control pins (7 and 9) again, and thus the device (1) is made ready for a second suturing with the mechanism transferred to either right or left of the first suturing.

Meanwhile, the stopper can be readjusted at the place where the device (1) is present or can be readjusted by removing the device (1) if required, and the suturing can be placed in the same manner by tracing the path in the first suturing. Adjustment processes of the stopper is done by pulling it out or pushing it in with gradual movements of 0.5 cm. After suturing in the localization having difficulties in access, suturing process for the other tissue with which the former tissue is joined can be successively repeated. If the second suturing process poses a challenge in access but is necessary for a different anatomical localization, the aforementioned processes would be applied by removing the needle (18) from the device (1) and attaching a new needle (18) to the later.

The movable jaw end (12) is integrated to the movable jaw (4) in the basic embodiment of the invention while the movable jaw end (12) is alternatively can be a movable jaw end attachment (20) that can be kept by being removed from the movable jaw (4) by use of the needle (18) in another embodiment of the invention. In this case, a new movable jaw attachment (20) can be used for another suturing on a different localization, as described above by being attached to the device (1) together with its needle (18) attached with the thread (19). The aim of this alternative is to achieve using again the said attachment (20) as a continuation of the first suturing within the phases of the operation.

In addition to that, thanks to the integration of the suturing device (1) according to the invention to the method of placing the mini sling that is used in the treatment of urinary incontinence in women, there have been achieved some improvements in the results obtained by the method of mini sling.

Based on the aspect of the invention, which is suturing on narrow and deep anatomical surfaces, use of the invention would be possible in the placement of minimal invasive sling, which is defined as the placement of a midurethral tape without using a transobturator path, and the similar operations. In this operation, which is known as mini sling, the stitches are passed through the anatomical triangulation points determined mutually in a symmetrical manner and then polipropilen is passed through the patch in a sling form in particular as well and thus, the patch would be secured to the desired localization in a suitable tension when the stitches are tied by considering the desired tension.

With a more detailed description, anchors in various forms are used in tissue fixation to the anatomical triangulation points determined at two sides of the midurethral patch in mini sling surgery. The thread attached onto the back of the anchors also holds on to the mini sling and the mini slings are placed under the urethra structure with a tubular form that exits from the urinary bladder, and symmetrically fixed to the tissues within the “narrow and deep localization” at the sides by these anchors. Up here, the most prominent drawback in the prior art is that this fixation process cannot be carried out in any case with the same effectiveness due to the reasons caused by the anchors.

By means of the suturing device (1) according to the invention, a stable fixation to the surfaces in which the anchors are placed can be achieved by suturing with the help of usage of the device (1) disclosed in the present description, and thus, a novel method of mini sling placement is provided.

Furthermore, the aspect of the device (1) of the invention that allows suturing through a challenging and single-impulse surface is used in mini sling surgeries.

Moreover, a surgical kit has been provided, which includes the suturing device (1) according to the invention for the mini sling surgery as well, and thus, the entire operation is enabled to be performed more successfully relative to the case in the prior art with the use of compatible devices.

In a similar way, different surgical kits containing the suturing device (1) according to the invention and equipped in accordance with the different operating methods are also possible to be provided.

1. A suturing device (1) developed to be used in repair of the tissue structures with access difficulties due to their narrow and deep localizations, which lack anatomical integrity due to ruptures, breakages or other various reasons, characterized in that it comprises;
at least two holding tabs (2) that allow the operator to grasp the suturing device (1) and a jaw assembly (3) to be opened and closed,
a jaw assembly (3) formed of a fixed jaw (5) containing a movable jaw (4) having one needle grasping slot (17) and one needle (18) on a jaw end (12), and one stopper partition (15) and one needle grasping slot (16) on a jaw end (14),
a fixed jaw arm (6) that enables the fixed jaw (5) to be controlled via the holding tab (2) and is coupled to the fixed jaw (5) through a carrying arm body (10),
a fixed jaw mechanism control pin (7) that allows the mechanism at the fixed jaw end (14) to be controlled by passing through the fixed jaw carrying arm (6), and also allows a needle (18) to which a thread (19) is attached to be transferred between the movable and fixed jaw needle grasping slots (17 and 16),
a fixed jaw carrying arm (8) that allows the movable jaw (4) to be controlled via the holding tab (2) and is coupled to the movable jaw (4) through the carrying arm body (10),
a movable jaw mechanism control pin (9) that allows the mechanism at the movable jaw end (12) to be controlled by passing through the movable jaw carrying arm (8), and also allows a needle (18) to which a thread (19) is attached to be transferred between the movable and fixed jaw needle grasping slots (17 and 16),
a carrying arm body (10) in which the fixed jaw carrying arm (6) and the movable jaw carrying arm (8) are joined,
a movable jaw base (11) and a fixed jaw base (13) that have a flat geometry so as to be closed on a horizontal plane when they correspond to each other,
a movable jaw end (12) and a fixed jaw end (14) that have a geometry so as to be closed by making an angle in the direction of the movable jaw base (11), relative to the movable jaw base (11) and the fixed jaw base (13) when they correspond to each other,
a stopper partition (15) located on the fixed jaw end (14) and to which a stopper can be attached,
a stopper that can be attached to the stopper partition (15) located on the fixed jaw end (14) and is used to allow the point to be sutured to be safe regarding the neurovascular vicinity based on the anatomical triangulation point,
a fixed jaw needle grasping slot (16) and a movable jaw needle grasping slot (17) that are located at the end portions of the movable and fixed jaws (4 and 5) which allows the needle (18) to which the thread (19) is attached to be secured in order to make the same ready for going in to the tissue, and
a needle (18) to which a thread is attached via the fixed jaw mechanism control pin (7) and the fixed jaw mechanism control pin.

2. Suturing device (1) according to claim 1, characterized in that; the movable jaw end (12) is integrated to the movable jaw (4).

3. Suturing device (1) according to claim 1, characterized in that; the movable jaw end (12) has a structure as a movable jaw attachment (20) that can be kept by removing from the movable jaw (4) with the needle (18) and used, when needed, by being attached to the movable jaw (4) with the needle (18) attached with the thread (19).

4. Suturing device (1) according to claim 1, characterized in that; the stopper whose length is determined based on the tissue it would go into, has a structure with flexible elasticity not including any sharp or cutting element.

5. Suturing device (1) according to claim 1, characterized in that; it is used in the method of mini sling placement in order to repair urinary incontinence in women.

6. A surgical kit provided to be used in different operating methods, and containing the suturing device (1) according to claim 1.

7. A surgical kit according to claim 6, characterized in that; it is provided to be used in the method of mini sling placement.
